(19) United States
(12) Patent Application Publication
(10) Pub. No.: US 2026/0297030 A1
(43) Pub. Date: Oct. 1, 2026

(54) DIPHENOXYLATE ARTIFICIAL HAPTEN, DIPHENOXYLATE ARTIFICIAL ANTIGEN, PREPARATION METHODS THEREFOR, AND USE THEREOF

(71) Applicant: Hangzhou Alltest Biotech Co., Ltd, Hangzhou (CN)

(72) Inventors: Haijian WANG, Hangzhou (CN); Yanpeng LU, Hangzhou (CN); Zhen WANG, Hangzhou (CN); Mengmeng XU, Hangzhou (CN); Cheng LIU, Hangzhou (CN)

(73) Assignee: Hangzhou Alltest Biotech Co., Ltd, Hangzhou (CN)

(21) Appl. No.: 19/234,573

(22) Filed: Jun. 11, 2025

(30) Foreign Application Priority Data
Mar. 28, 2025 (CN) 202510384517.3

(51) Int. Cl. C07D 211/34; C07K 16/44; G01N 33/543; G01N 33/94

(52) U.S. Cl. CPC C07D 211/34; C07K 16/44; G01N 33/54387; G01N 33/9486

(57) ABSTRACT

Provided in the present disclosure are a diphenoxylate artificial hapten, a diphenoxylate artificial antigen, preparation methods therefor, and use thereof, relating to the technical field of immunological testing. The hapten has a structure represented by formula I. The present disclosure further provides the preparation method for the hapten, the artificial antigen formed by covalent conjugation of the artificial hapten with a carrier protein, and an antibody obtained by immunization with the artificial antigen; and develops a diphenoxylate detection device, such as a test strip and a test card.

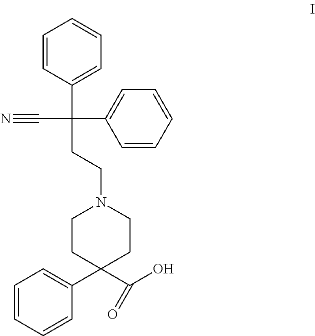 [SMILES: N#CC(CCN1CCC(C(=O)O)(c2ccccc2)CC1)(c1ccccc1)c1ccccc1]

### CROSS-REFERENCES TO RELATED APPLICATION

The present application claims the benefit of the priority of the Chinese patent application with the application No. 202510384517.3, filed to the China National Intellectual Property Administration on Mar. 28, 2025, the entire content of which is incorporated in this application by reference.

### TECHNICAL FIELD

The present disclosure belongs to the technical field of immunological testing, and particularly relates to a diphenoxylate artificial hapten, a diphenoxylate artificial antigen, preparation methods therefor, and use thereof.

### BACKGROUND

Diphenoxylate (DIP) is an artificially synthesized piperidine derivative with the chemical name 1-(3,3-Diphenyl-3-cyanopropan-1-yl)-4-phenylpiperidine-4-carboxylate, the chemical formula C₃₀H₃₂N₂O₂, and the structural formula shown below:

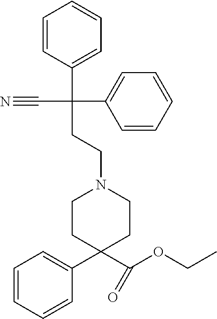 [SMILES: CCOC(=O)C1(c2ccccc2)CCN(CCC(C#N)(c2ccccc2)c2ccccc2)CC1]

Diphenoxylate has similar effects to opioid active drugs and can therefore be used for treating withdrawal reactions from opioids of abuse. Some drug abusers have abused heroin before abusing compound diphenoxylate tablets. The primary initial reason for abuse is drug rehabilitation or controlling withdrawal symptoms. The second reason is as a substitution for heroin. Overdosing on diphenoxylate can cause acute poisoning symptoms such as dyspnea and lethargy, which are life-threatening. Some abusers experience withdrawal symptoms like diarrhea after sudden discontinuance. Consequently, diphenoxylate has been listed in Schedule II psychotropic substances under control.

Due to the instability of diphenoxylate prototypes and the rapid metabolism of diphenoxylate in the human body, there is a lack of stable raw material with physicochemical properties similar to the diphenoxylate prototype in the process of the development of related test products. The raw material is applicable to coating substrates for enzyme-linked immunosorbent assay (ELISA), coating antigens in colloidal gold immunochromatography, and immunogens in antibody preparation. In clinical drug monitoring, the rapid on-site test for the diphenoxylate prototype can more directly and accurately reflect recent usage of the drug. Thus, developing a stable raw material with physicochemical properties similar to the diphenoxylate prototype is particularly crucial.

At present, the test methods for diphenoxylate include gas chromatography, capillary electrophoresis, and high-performance liquid chromatography. However, these methods involve complex procedures and time-consuming analysis, limiting their disclosure in large-batch biological sample analysis. The liquid chromatography-electrospray ionization-mass spectrometry (LC-ESI-MS) assay developed based on protein precipitation has a relatively high lower limit of quantification and cannot provide sufficient sensitivity for diphenoxylate detection in the human body.

To cope with the increasingly severe situations in drug abuse prevention and control, it is an urgent need to develop an immunochromatographic product capable of rapidly, accurately, conveniently, and efficiently collecting and testing human urine samples and preliminarily analyzing diphenoxylate residue levels or drug abuse in the human body within a relatively short time. The high-efficiency synthesis of an artificial antigen is the prerequisite and key to ensuring immunoassays.

### SUMMARY

Aiming at the defects in the prior art, an objective of the present disclosure is to improve the specificity and sensitivity of diphenoxylate immunological detection. The present disclosure provides a diphenoxylate artificial hapten, a diphenoxylate artificial antigen, preparation methods therefor, and use thereof.

In a first aspect, the present disclosure provides a diphenoxylate artificial hapten, having a structure represented by formula I:

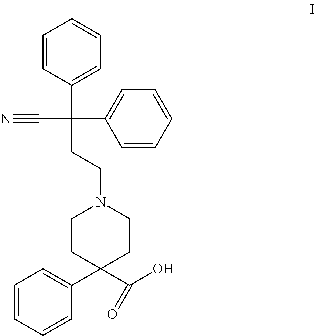 [SMILES: N#CC(CCN1CCC(C(=O)O)(c2ccccc2)CC1)(c1ccccc1)c1ccccc1]

In a second aspect, the present disclosure provides a preparation method for the diphenoxylate artificial hapten, including the following steps:

dissolving diphenoxylate in a mixed organic solvent; adding an aqueous sodium hydroxide solution; stirring at a room temperature for a hydrolysis reaction; adjusting pH to 4-5 after the reaction is completed; and extracting to remove the solvent and purifying, to obtain the diphenoxylate artificial hapten.

Preferably, the mixed organic solvent is a mixed solvent of tetrahydrofuran and anhydrous methanol.

Preferably, a normality of the aqueous sodium hydroxide solution is 1 N.

In the hydrolysis reaction, any relevant techniques applicable to the conditions of the hydrolysis reaction of diphenoxylate prototype compounds can be adopted. To improve hydrolysis efficiency, preferably, a volume of the tetrahydrofuran is generally 8-20 mL per gram of the diphenoxylate; preferably, a volume of the anhydrous methanol is generally 10-20 mL per gram of the diphenoxylate; preferably, a molar ratio of the diphenoxylate to NaOH in the IN aqueous sodium hydroxide solution is 1:10 to 50; preferably, time for the hydrolysis reaction is 4-6 hours; and preferably, pH is adjusted with 1 N hydrochloric acid.

In extraction and purification processes, any relevant techniques applicable to the conditions of the extraction of the reaction solution can be adopted. To simplify procedures, preferably, an extraction agent is selected from one of dichloromethane, 1,1-difluoro-2-chloroethane, dimethyl carbonate, and propylene glycol methyl ether acetate, more preferably dichloromethane; preferably, a chromatographic solution for thin-layer chromatography is a mixed solvent of ethanol with a volume fraction of 95%, 1,4-dioxane, dichloromethane, and ammonia solution in a volume ratio of 8:1:10:1, with product Rf value=0.5; and preferably, the ammonia solution has a mass fraction of 25% to 28%.

In a third aspect, the present disclosure provides a diphenoxylate artificial antigen, formed by covalent conjugation of the diphenoxylate artificial hapten with a carrier protein, and having a structure represented by formula II:

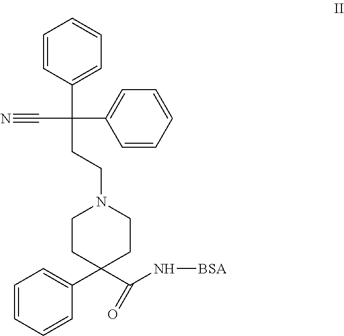

Preferably, the carrier protein is a bovine serum albumin (BSA).

In a fourth aspect, the present disclosure provides a preparation method for the diphenoxylate artificial antigen, including the following steps:
mixing and dissolving the diphenoxylate artificial hapten, an ester activator, and a catalyst in an organic solvent; stirring for reaction at 0-5° C. for 2-4 hours; collecting a supernatant to be added into a bovine serum albumin solution; standing at 3-5° C. overnight; and carrying out dialysis and centrifugation, to obtain the diphenoxylate artificial antigen represented by formula II.

Preferably, the ester activator is selected from one of isoethyl chloroformate, isobutyl chloroformate, butyl chloroformate, and benzyl chloroformate, more preferably isoethyl chloroformate.

Preferably, the catalyst is triethylamine or N-methylpyrrolidone, more preferably triethylamine. The above catalysts indirectly facilitate the non-covalent binding or covalent modification reactions between small molecules and BSA by adjusting a polar environment of a reaction system.

Preferably, the organic solvent is selected from N,N-dimethylformamide or N,N-dimethylacetamide, more preferably N,N-dimethylformamide.

Preferably, a molar ratio of the diphenoxylate artificial hapten to the ester activator to the catalyst is 1:1.5 to 2.5:1 to 1.5, more preferably 1:2:1.

Preferably, a volume of the organic solvent is 30-80 mL per gram of the diphenoxylate artificial hapten represented by formula I, more preferably 50 mL/g.

Preferably, the bovine serum albumin solution is prepared by dissolving the bovine serum albumin in a 0.01 M PBS buffer with pH=7.2 to 7.4.

Preferably, a concentration of the bovine serum albumin solution is 5 mg/mL.

Preferably, a volume ratio of the supernatant to the bovine serum albumin solution is 1:5 to 6.

In a fifth aspect, the present disclosure provides a diphenoxylate antibody, which is generated in response to the diphenoxylate artificial antigen, and specifically obtained by animal immunization, wherein animals include BALB/c mice and New Zealand white rabbits.

Tests found that a titer of diphenoxylate monoclonal antibody prepared by immunizing the BALB/c mice with the diphenoxylate artificial antigen was 1:128,000, and a titer of an immune serum obtained by immunizing the New Zealand white rabbits with the diphenoxylate artificial antigen was 1:4,000. The New Zealand white rabbits were first immunized with the diphenoxylate artificial antigen at 0.25 mg/kg every 7 days, and the antibody titer reached a maximum value after the third immunization. After the fourth immunization, immunization was continuously carried out at 0.1 mg/kg for 6 months, without decrease in antibody titer. This shows that the diphenoxylate artificial antigen of the present disclosure has good immunogenicity, and can produce diphenoxylate antibodies with high affinity, high sensitivity, and strong specificity through immunization. The diphenoxylate antibodies can be used for immunodetection and analysis of diphenoxylate.

Preferably, the diphenoxylate monoclonal antibody has a high titer and specificity.

In a sixth aspect, the present disclosure provides use of the diphenoxylate antibody in the detection of diphenoxylate.

Cross-interference substance tests found that diphenoxylate test strips have good specificity and certain anti-interference capabilities. When other substances, including opioids, fentanyl, and analogs thereof, are present in samples, they do not affect the detection of the diphenoxylate test strips.

In a seventh aspect, the present disclosure provides a diphenoxylate detection device, including the antibody. Preferably, the detection device is a test kit, a test strip, a test plate, or a test card.

In summary, compared with the prior art, the present disclosure has the following beneficial effects:

First, according to the present disclosure, a carbonyl group and a carbon chain terminus in a diphenoxylate prototype compound are modified into carboxyl terminuses, thereby facilitating the linking to macromolecular proteins. Moreover, the stability and solubility of the modified diphenoxylate artificial hapten are improved to a certain extent compared with the diphenoxylate prototype. The prepared diphenoxylate artificial hapten is not prone to degradation within a temperature range of −20° C. to 55° C. and a pH range of 5 to 11, facilitating large-scale preparation of artificial antigens and reducing production costs.

Second, the diphenoxylate prototype has multiple benzene ring structures, which are unsuitable for linking to macromolecular proteins. Introducing a linker arm at the modification site can preserve the characteristic structure of diphenoxylate to the greatest extent. Moreover, the modification site is as far away from the characteristic functional group of diphenoxylate as possible, such that the characteristic part thereof is exposed as far as possible, so as to avoid interference with a specific antigenic epitope, and to be recognized by an immune organism as far as possible. Additionally, it prevents the immunogenicity of the synthesized artificial antigen from being affected by the main structure of diphenoxylate being too close to macromolecular proteins, which is obscured by the spatial conformation of protein folding.

Third, the test strips, test kits, and detection devices prepared using the diphenoxylate artificial hapten, the diphenoxylate artificial antigen, and the diphenoxylate antibody provided by the present disclosure can rapidly, accurately, conveniently, and efficiently collect and test human urine samples, and simultaneously analyze diphenoxylate drug residue levels or drug abuse in the human body within a relatively short time. Compared with conventional instrumental test methods, they have advantages such as low cost, simple operation, suitability for large-scale initial sample screening in practical test scenarios, and accurate and reliable results.

### BRIEF DESCRIPTION OF THE DRAWINGS

FIG. 1 shows high-performance liquid chromatography (HPLC) of a diphenoxylate artificial hapten.

FIG. 2 shows high-resolution electrospray ionization-mass spectrometry (ESI-MS) of a diphenoxylate artificial hapten.

FIG. 3 shows an ultraviolet absorption spectra of diphenoxylate artificial antigen II before and after preparation.

FIG. 4 shows a diphenoxylate test strip.

FIG. 5 shows a diphenoxylate test plate.

FIG. 6 shows a diphenoxylate test card.

### DETAILED DESCRIPTION OF THE EMBODIMENTS

The following will describe the implementation solutions of the present disclosure in detail in conjunction with the examples.

### Example 1

A specific preparation method for a diphenoxylate artificial hapten was as follows:

600 mg (1.33 mmol) of diphenoxylate was dissolved with 6 mL tetrahydrofuran and 9 mL anhydrous methanol in a 50 mL round-bottom flask. 40 μL (39.9 mmol) of 1 N aqueous sodium hydroxide solution was added. Stirring for a reaction was carried out at a room temperature for 0.5 hours. The pH of the solution after the reaction was adjusted to 5 with 1 N hydrochloric acid. 5 mL Dichloromethane was added to extract the solution. The resulting mixed solution was filtered and dried to remove the solvent to obtain an oily substance, i.e., a crude diphenoxylate artificial hapten. A chromatographic eluent was prepared from 95% ethanol (by volume fraction), 1,4-dioxane, dichloromethane, and 28% ammonia solution (by volume fraction) in a volume ratio of 8:1:10:1. The crude diphenoxylate artificial hapten is subjected to chromatographic purification, to obtain 542 mg of a diphenoxylate artificial hapten.

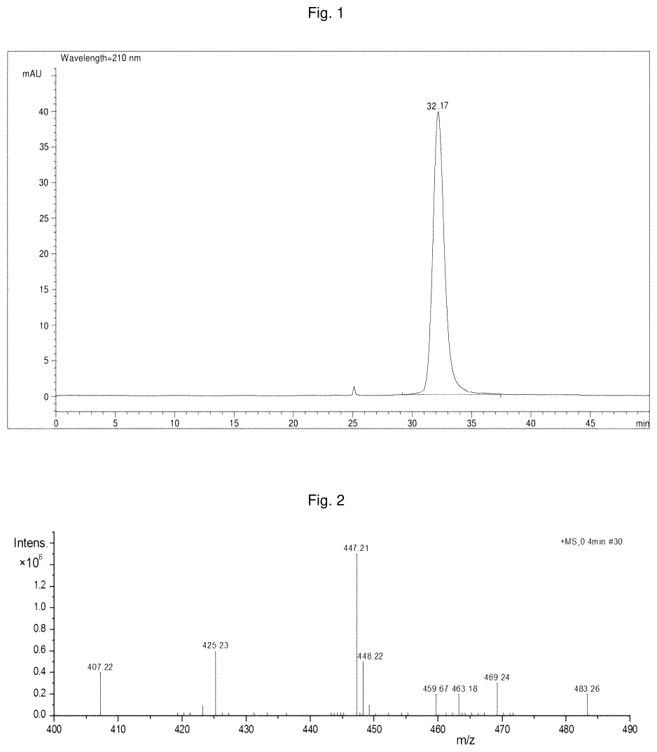

The liquid chromatogram of diphenoxylate artificial hapten I is shown in FIG. 1 (with an ultraviolet detector with a wavelength in nm). The results show that the diphenoxylate artificial hapten obtained by purification has a purity reaching above 98%, fully meeting the requirements for artificial antigen preparation.

The high-resolution ESI-MS of diphenoxylate artificial hapten I is shown in FIG. 2. From FIG. 2, it can be seen that the characteristic peaks of the obtained diphenoxylate artificial hapten are 425.23, 447.21, 448.22, 463.18, 469.24, and 483.26, corresponding to [M+H]+, [M+Na]+, [M+Na+H]+, [M+K]+, [M+HCOO]−, and [M+CH3COO]− respectively, wherein [M+H]+ has the peak of 425.23, which matches the theoretical molecular weight of 424 for the diphenoxylate artificial hapten. Based on the above data, it can be preliminarily confirmed that the final compound obtained through the steps is diphenoxylate artificial hapten I designed in the present disclosure.

### Example 2

A specific preparation method for a diphenoxylate artificial antigen was as follows:

542 mg (1.28 mmol) of diphenoxylate artificial hapten I was placed into a 50 mL round-bottom flask. 20 mL of N,N-dimethylformamide (DMF) was added. 166.7 μL (1.2 mmol) of triethylamine was then added. The flask was placed in an ice bath for stirring for 30 min. 156.1 μL (1.2 mmol) of isobutyl chloroformate was then added. Stirring for reaction was carried out in the ice bath for 2 hours. After the reaction was completed, centrifugation was carried out. Approximately 19.4 mL of a supernatant was collected for later use. 500 mg of bovine serum albumin was weighed and dissolved in 100 mL of 0.01 M PBS buffer with pH=7.2 to 7.4 to prepare a bovine serum albumin solution. The supernatant is mixed with the bovine serum albumin solution. The mixture stands at 4° C. overnight, to obtain a diphenoxylate artificial antigen mixed solution.

The diphenoxylate artificial antigen mixed solution was transferred into a dialysis bag and dialyzed three times with the above PBS buffer, for 2 hours each time. After dialysis, polyethylene glycol 8000 powder was sprinkled on the outer side of the dialysis bag for concentration. The concentration was stopped when the volume of the solution in the dialysis bag was reduced to one-third of the original volume. The solution was poured out and then centrifuged. A supernatant was collected to obtain diphenoxylate artificial antigen II: a diphenoxylate-bovine serum albumin conjugate.

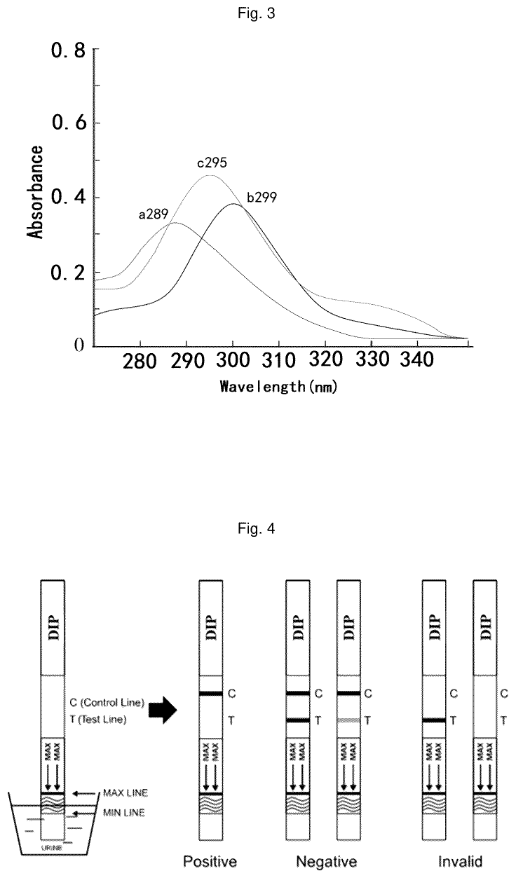

The ultraviolet absorption spectra of diphenoxylate artificial antigen II before and after preparation are shown in FIG. 3. In FIG. 3, curve a represents the ultraviolet scan of diphenoxylate artificial hapten I, curve b represents the ultraviolet scan of diphenoxylate artificial antigen II, and curve c represents the ultraviolet scan of bovine serum albumin. The maximum absorption wavelength of diphenoxylate artificial hapten I is 289 nm, the maximum absorption wavelength of diphenoxylate artificial antigen II is 299 nm, and the maximum absorption wavelength of bovine serum albumin is 295 nm. Compared with diphenoxylate artificial hapten I and bovine serum albumin, the maximum absorption wavelength of diphenoxylate artificial antigen II shows a significant change, indicating that diphenoxylate artificial hapten I is successfully conjugated with bovine serum albumin.

### Example 3

A specific preparation method for an antibody capable of binding to diphenoxylate was as follows:

### 3.1 Monoclonal Antibody

Four 6-week-old BALB/c mice were immunized with a diphenoxylate artificial antigen as an immunogen, via multiple-site subcutaneous injection on the back at an immunization dose of 100 μg per mouse, with immunization every 2 weeks. The primary immunization used the immunogen and an equal volume of Freund's complete adjuvant, and the second and third immunizations used the immunogen and an equal volume of Freund's incomplete adjuvant. Seven days after the third immunization, blood was collected from the submandibular vein, and serums were collected for titer determination. Three days before fusion, a pure antigen was injected intraperitoneally for booster immunization.

The four immunized mouse serums were diluted at 1:4,000, 1:8,000, and 1:16,000 respectively. Using a diphenoxylate artificial antigen as a coating antigen, the serum titers were determined by indirect ELISA. Fusion could be carried out when titer >104. Three days after the booster immunization, mouse splenocytes were collected and fused with SP2/0 myeloma cells. After the fusion, supernatants were collected and tested by the above indirect ELISA. Then, positive hybridoma cells were subcloned by limiting dilution and checkerboard titration. Whether subcloning was successful was tested by the established indirect ELISA. The cells in positive wells were sequentially cloned three times using the above method. Positive hybridoma wells with high OD values, good cell viability, and stable antibody secretion were selected for extended culture and timely cryopreservation.

7-week-old BALB/c mice were injected intraperitoneally with 0.5 mL sterile liquid paraffin. After 7-10 days, each mouse was injected intraperitoneally with 1.0 mL of hybridoma cells (1×10⁶ cells/mL). 14 Days later, ascitic fluid was collected and centrifuged to collect supernatants. Purification was carried out using a Protein L Resin kit. The concentration of the purified ascitic fluid was determined with a BCA Protein Assay Kit. Titer determination was carried out on the monoclonal antibodies according to the above indirect ELISA. The results show that the prepared diphenoxylate monoclonal antibody has a titer of 1:128,000.

### 3.2 Polyclonal Antibody

The concentration of a diphenoxylate artificial antigen was adjusted to 0.5 mg/mL. Blood was collected from the marginal ear vein before primary immunization, and negative serums were separated. For the primary immunization, 2 mL of an antigen solution (1 mg antigen) and an equal volume of Freund's complete adjuvant were mixed, oscillated, and emulsified. The completely emulsified mixed solution was inoculated subcutaneously to the multiple sites of the rabbit's neck and back. After the primary immunization, the diphenoxylate artificial antigen and the Freund's incomplete adjuvant were mixed in equal proportions every 10 days and completely emulsified for booster immunization. The secondary immunization involved 1 mg antigen injection. The third and fourth immunizations involved 0.5 mg antigen injection. Cardiac blood was collected 10 days after the fourth immunization. After standing at a room temperature for 1 hour, centrifugation was carried out at 4° C. and 5,000 rpm for 20 min to collect polyclonal antibody serums. Titer determination was carried out on the polyclonal antibodies according to the above indirect ELISA. The results show that the prepared diphenoxylate polyclonal antibody has a titer of 1:64,000.

### Disclosure Example 1: Preparation of Diphenoxylate Colloidal Gold Immunochromatographic Test Strip/Plate/Card

### (1) Preparation of Gold-Labeling Pad

A diphenoxylate monoclonal antibody-colloidal gold particle complex and a goat anti-rabbit antibody-colloidal gold particle complex were taken and diluted together in a phosphate buffer containing bovine serum gamma globulin. Trehalose and sucrose were added. After uniform mixing, the mixture was sprayed onto a conjugate pad to obtain the gold-labeling pad.

### (2) Treatment of Nitrocellulose Membrane

A 0.2 mg/mL diphenoxylate-protein conjugate antigen and 0.2 mg/mL rabbit immunoglobulin G were respectively diluted with a phosphate buffer to obtain coating solutions. The test line, i.e., “T line”, was obtained by linear sampling of a diphenoxylate-bovine serum albumin conjugate antigen onto the nitrocellulose membrane. The control line was obtained by linear sampling of a goat anti-rabbit antibody.

### (3) Treatment of Sample Pad

A glass fiber solution was prepared and coated onto blank glass fiber, which was then dried at 37° C. for 12 hours, to obtain the sample pad, with a coating dose of 0.4 mL/cm².

The glass fiber solution included: pure water, Tris (12 mg/mL), polyvinylpyrrolidone (10 mg/mL), S-9 (10 mg/mL), sodium caseinate (1 mg/mL), sodium cholate (5 mg/mL), and sodium azide (0.2 mg/mL), adjusted to pH of 8.0.

### (4) Assembly of Immunochromatographic Test Strip

As shown in FIG. 4, a sample pad, a gold-labeling pad, a nitrocellulose membrane, and an absorbent pad were pasted to a plastic plate sequentially from bottom to top. The connected parts were overlapped by approximately 1.5 mm. When a test sample was dropwise added to the sample pad, the sample was subjected to chromatography upward by the capillary effect to complete the reaction.

### (5) Result Interpretation

Positive result: a red line appeared at the C line (Control Line) in the test strip display zone. Negative result: red lines appeared at both the C line (Control Line) and T line (Test Line) in the test strip display zone. Invalid result: no red lines appeared at either the C line (Control Line) or T line (Test Line) in the test strip display zone, or a red line appeared only at the T line.

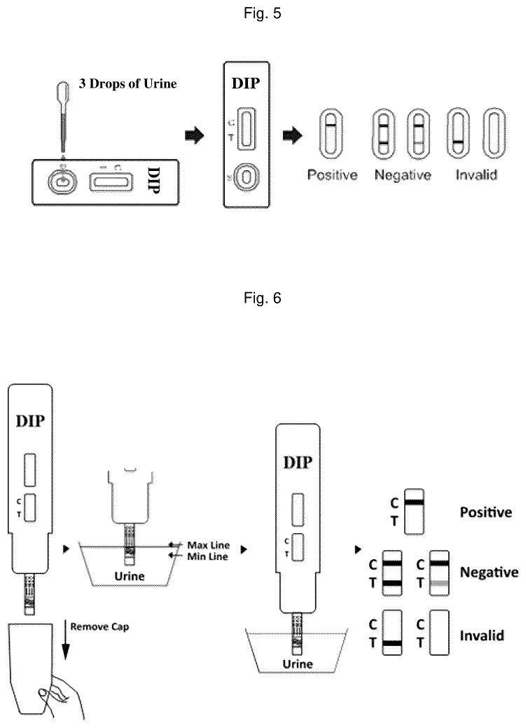

By placing the test strips in different housings, test plates and test cards can be obtained, with structures shown in FIG. 5 and FIG. 6, respectively.

### Test Example 1: Test on Sensitivity of DIP Test Strips

The sensitivity was determined and analyzed using negative urine specimens at drug-free urine (negative), −50% test threshold, −25% test threshold, +25% test threshold, +50% test threshold, and +300% test threshold. These standards were randomized and coded. The results were verified by liquid chromatography-mass spectrometry (LC/MS). Each standard was tested in 10 replicates. The test results of the test strips were visually interpreted 5 minutes after the sample disclosure. The results are shown in Table 1, where “+” represents positive; “−” represents negative.

TABLE 1
Test on sensitivity of DIP test strips

| DIP content | Test result |
|---|---|
| Drug-free urine | −,−,−,−,−,−,−,−,−,− |
| 250 ng/mL | −,−,−,−,−,−,−,−,−,− |
| 375 ng/mL | −,−,−,−,−,−,−,−,−,+ |
| 500 ng/mL | −,−,−,−,−,+,+,+,+,+ |
| 625 ng/mL | +,+,+,+,+,+,+,+,+,− |
| 750 ng/mL | +,+,+,+,+,+,+,+,+,+ |
| 1500 ng/mL | +,+,+,+,+,+,+,+,+,+ |

The results show that the test threshold of the DIP test strips is approximately 500 ng/ml.

### Test Example 2: Evaluation on Accuracy of DIP Test Strips

For the accuracy evaluation, 150 urine samples were used, including 50 positive urine specimens and 100 negative urine specimens, all confirmed by LC/MS. After being randomized, the urine samples were tested by the DIP test strips. The results were interpreted 5 minutes after sample disclosure. The test results are shown in Table 2.

TABLE 2
Evaluation on accuracy of DIP test strips

| Test method | | LC/MS | | |
|---|---|---|---|---|
| | Result | Positive | Negative | Total |
| DIP test | Positive | 48 | 3 | 51 |
| strips | Negative | 2 | 97 | 99 |

| | | | |
|---|---|---|---|
| Total | 50 | 100 | 150 |

Relative Sensitivity=48/50=96.0% (86.29% to 99.51%)
Relative Specificity=97/100=97.0% (91.48% to 99.38%)
Overall Consistency=145/150=96.7% (92.39% to 98.91%)

From the above data, it can be seen that the DIP detection test strips have a relative sensitivity of 96.0%, a relative specificity of 97.0%, and an overall consistency of 96.7%.

### Test Example 3: Test on Effects of Urine Specific Gravity on Accuracy of DIP Test Strips

This example used a urine specific gravity refractometer to determine urine specific gravity. A total of 15 urine samples were used, with specific gravity falling within the following ranges: (1) specific gravity >1.03; (2) specific gravity being 1.012 to 1.03; (3) specific gravity <1.012. Each urine sample was divided into three portions: (1) negative urine; (2) DIP was added into the urine sample to 250 ng/ml; (3) DIP was added into the urine sample to 750 ng/ml. Repeated tests were conducted on the above urine samples using DIP test strips. The test results are shown in Table 3, where “+” represents positive; “−” represents negative.

TABLE 3
Effects of urine specific gravity on accuracy of DIP test strips

| Urine | Specific | | Negative | | DIP 250 | | DIP 750 | |
|---|---|---|---|---|---|---|---|---|
| sample | gravity | Replicates | urine | | ng/ml | | ng/ml | |
| 1 | 1.006 | 2 | − | − | − | − | + | + |
| 2 | 1.008 | 2 | − | − | − | − | + | + |
| 3 | 1.009 | 2 | − | − | − | − | + | + |
| 4 | 1.011 | 2 | − | − | − | − | + | + |
| 5 | 1.012 | 2 | − | − | − | − | + | + |
| 6 | 1.014 | 2 | − | − | − | − | + | + |
| 7 | 1.018 | 2 | − | − | − | − | + | + |
| 8 | 1.022 | 2 | − | − | − | − | + | + |
| 9 | 1.027 | 2 | − | − | − | − | + | + |
| 10 | 1.030 | 2 | − | − | − | − | + | + |
| 11 | 1.031 | 2 | − | − | − | − | + | + |
| 12 | 1.036 | 2 | − | − | − | − | + | + |
| 13 | 1.037 | 2 | − | − | − | − | + | + |
| 14 | 1.039 | 2 | − | − | − | − | + | + |
| 15 | 1.041 | 2 | − | − | − | − | + | + |

The results show that the normal variation range of urine specific gravity does not affect the test results of the DIP test strips.

### Test Example 4: Test on Effects of Urine pH on Accuracy of DIP Test Strips

This example used 15 urine samples with different pH. Each urine sample was divided into three portions and respectively placed into three glass containers. DIP was added to two of the containers to achieve final sample concentrations of 250 ng/ml and 750 ng/ml, respectively, while the third served as a control group (negative urine). The results of all the samples were interpreted 5 minutes and 10 minutes after testing. The test results are shown in Table 4, where “+” represents positive; “−” represents negative.

TABLE 4
Effects of urine pH on accuracy of DIP test strips

| | Negative urine | | −50% test threshold | | +50% test threshold | |
|---|---|---|---|---|---|---|
| pH | 5 min | 10 min | 5 min | 10 min | 5 min | 10 min |
| 4.05 | − | − | − | − | + | + |
| 4.73 | − | − | − | − | + | + |
| 5.36 | − | − | − | − | + | + |
| 5.51 | − | − | − | − | + | + |
| 6.24 | − | − | − | − | + | + |
| 6.94 | − | − | − | − | + | + |
| 7.17 | − | − | − | − | + | + |
| 7.55 | − | − | − | − | + | + |
| 8.85 | − | − | − | − | + | + |
| 9.12 | − | − | − | − | + | + |

The results show that the changes in urine pH do not affect the test results of the DIP test strips.

### Test Example 5: Test on Effects of Different Chemical Substances on Accuracy of DIP Test Strips

To evaluate the cross-reactivity of the DIP test strips with different chemical substances when testing drug-free urine, in this example, different compounds were added into urine samples at a concentration of 100 μg/mL, and testing was carried out using DIP test strips. The results showed that none of the compounds shown in Table 5 caused cross-interference with the DIP test strips.

TABLE 5
Substances without cross-interference with DIP test strips

| | | |
|---|---|---|
| (+/−)-Phenylpropanolamine | Diphenhydramine | Olopatadine |
| (d,l-Norephedrine) | | |
| 3,4-Methylenedioxyethylamphetamine | Disulfiram | Omeprazole |
| 4-Amino-3-phenylbutric | Doxepin | Orphenadrine |
| 4-Fluoromethcathinone | Doxycycline | Oseltamivir phosphate |
| Acetamidophenol (paracetamol) | Doxylamine | Oxalic acid |
| Acetophenetidin (phenacetin) | Efavirenz (Sustiva) | Oxolinic acid |
| Acetylsalicylic acid (aspirin) | Emtricitabine | Oxymetazoline |
| Albumin | Enoxaparin sodium | Oxymorphone |
| Alimemazine Tartrate | Estazolam | Pantoprazole Sodium |
| | | Enteric-Coated |
| Allopurinol | Ethyl-p-aminobenzoate | Papaverine |
| | (Benzocaine) | |
| Alprazolam | Etoricoxib | Paroxetine |
| Aminopteridine | Fenoterol hydrobromide | Perphenazine |
| AminopyrineAminophenazone | Fentanyl | Phenothiazine |
| Amitriptyline | Fexofenadine | Pholcodine |
| Amobarbital | Flucloxacillin sodium | Pimecillin |
| Amoxapine | Fluoxetine | Pramipexol |
| Amoxcillin | Furosemide | Prazosin |
| Ampicillin | Gatifloxacin | Prednisolone |
| Ascorbic acid L-Ascorbic acid | Guanfacine | Pregablin |
| Atomoxetine | Haloperidol | Procaine |
| Atorvastatin | Heroin | Propoxyphene |
| Atropine | Hexobarbital | Protopine |
| Beclometasone dipropionate | Hydralazine | Pseudoephedrine |
| Benzilic acid | Hydroxyphenylacetic acid | Pyrazinamide |
| Benzoylecgonine | Hydroxyzine | R(+)-Cathinone HCl |
| Bilirubin | Imipramine | R,R(−)-Pseudoephedrine |
| Biperiden | Isoniazid | Ritalin acid |
| Bisoprolol Fumarate | Isotretinoin | Rivaroxaban |
| Bisulepini | Kanamycin | Rosuvastatin Calcium |
| Bromazepam | Ketoprofen | S(−)-Cathinone HCl |
| Bromhexine | Lactulose | S,S(+)-Pseudoephedrine |
| Budesonide | Lamotrigine | Salbutamol |
| Bupropione | Lansoprazole | Salmeterol |
| Caffeine | Levomepromazine | Sertraline |
| Candesartan | Lidocaine | Sodium 2-Propylvalerate |
| Cannabidiol | Lisdexamfetamine | Sodium chloride |
| Carbamazepine | Loperamide | Sulfasalazine |
| Carbamide | Loratadine | Tenofovir alafenamide |
| | | fumarate |
| Carvedilol | Losartan potassium | Terbutaline |
| Cefalexin | Melatonine | Thebaine |
| Cetirizine | Melbine (DMBG) | Tianeptine |
| Chloral hydrate | Methadone | Tinidazole |
| Chloramphenicol | Methamphetamine | Tolbutamide |
| Cholesterol | Methylone | Tramadol hydrochlorid |
| Citalopram | Methylphenidate | Trazodone |
| Clonazepam | Metronidazole | Triazolam |
| Clonidine | Mexiletine | Trihexyphenidyl |
| Clozapine | Mirtazapine | Tyramine |
| Cocaine | Moxifloxacin | Uric acid |
| Collar hydroxyhippuric acid | Narcotine | Valproate |
| Cyclobenzaprine | N-Desmethylolanzapin | Venlafaxine |
| d,l-Tryptophan | Nitrazepam | Verapamil |
| Darunavir | Norcodeine | Vitamin B12 |
| Desipramine | Norfloxacin | Xylazine |
| Dexamethasone | N-Vanillylnonanamide | Zaleplon |
| Diazepam | o-Desmethyl-cis-tramadol | Zomepirac |
| Diclofenac sodium salt | Ofloxacin | Zopiclone |
| Diflunisal | Olanzapine | Δ9-Tetrahydrocannabinol |
| Dihydrocodeine | | |

The results show that the DIP test strips have high specificity and certain anti-interference capabilities. When other substances, including opioids, fentanyl, and analogs thereof, are present in the samples, they do not affect the detection of the DIP test strips.

The above examples describe the basic principles, main features, and advantages of the present disclosure, merely illustrating the technical aspects of the present disclosure rather than limiting the scope of protection of the present disclosure. Any modifications made based on the above examples shall fall within the scope of protection of the present disclosure.

What is claimed is:

1. A diphenoxylate artificial hapten, having a structure represented by formula I:

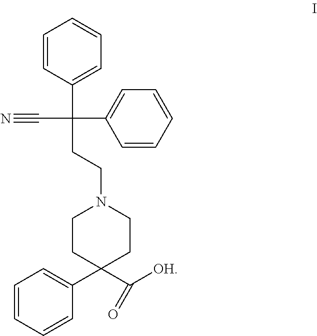 [SMILES: N#CC(CCN1CCC(C(=O)O)(c2ccccc2)CC1)(c1ccccc1)c1ccccc1]

2. A preparation method for the diphenoxylate artificial hapten according to claim 1, comprising the following steps:
dissolving diphenoxylate in a mixed organic solvent; adding an aqueous sodium hydroxide solution; stirring at a room temperature for a hydrolysis reaction; adjusting pH to 4-5 after the reaction is completed; and extracting to remove the solvent and purifying, to obtain the diphenoxylate artificial hapten.

3. The preparation method according to claim 2, wherein the mixed organic solvent is a mixed solvent of tetrahydrofuran and anhydrous methanol, wherein a volume of the tetrahydrofuran is 8-20 mL per gram of the diphenoxylate, and a volume of the anhydrous methanol is 10-20 mL per gram of the diphenoxylate.

4. The preparation method according to claim 2, wherein a normality of the aqueous sodium hydroxide solution is 1 N; and a molar ratio of the diphenoxylate to NaOH in the aqueous sodium hydroxide solution is 1:10 to 50.

5. A diphenoxylate artificial antigen, formed by covalent conjugation of the diphenoxylate artificial hapten according to claim 1 with a carrier protein, and having a structure represented by formula II:

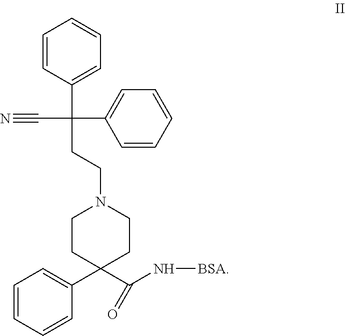

6. A preparation method for the diphenoxylate artificial antigen according to claim 5, comprising the following steps:
mixing and dissolving the diphenoxylate artificial hapten, an ester activator, and a catalyst in an organic solvent; stirring for reaction at 0-5° C. for 2-4 hours; collecting a supernatant to be added into a bovine serum albumin solution; standing at 3-5° C. overnight; and carrying out dialysis and centrifugation, to obtain the diphenoxylate artificial antigen represented by formula II.

7. The preparation method according to claim 6, wherein the ester activator is selected from one of isoethyl chloroformate, isobutyl chloroformate, butyl chloroformate, and benzyl chloroformate; the catalyst is triethylamine or N-methylpyrrolidone; and the organic solvent is selected from N,N-dimethylformamide or N,N-dimethylacetamide.

8. The preparation method according to claim 6, wherein
a molar ratio of the diphenoxylate artificial hapten to the ester activator to the catalyst is 1:1.5 to 2.5:1 to 1.5; and a volume ratio of the supernatant to the bovine serum albumin solution is 1:5 to 6.

9. A diphenoxylate antibody, obtained by animal immunization with the diphenoxylate artificial antigen according to claim 5.

10. A diphenoxylate detection device, comprising the antibody according to claim 9.
